(19) United States
(12) Patent Application Publication
(10) Pub. No.: US 2006/0275894 A1
(43) Pub. Date: Dec. 7, 2006

(54) Hydrogen producing apparatus utilizing excess heat from an industrial facility

(76) Inventors: Mitchell S. Felder, Hermitage, PA (US); Justin Felder, Hermitage, PA (US); Harry R. Diz, Erie, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/444,027

(22) Filed: May 31, 2006

Related U.S. Application Data
(60) Provisional application No. 60/686,008, filed on May 31, 2005.

(51) Int. Cl. C12M 3/00

(52) U.S. Cl. 435/289.1

(57) ABSTRACT

The present invention provides a hydrogen production apparatus, where a bioreactor is combined with an industrial facility such that the industrial facility heats an organic feed material prior to conveyance of the organic feed material into the bioreactor. The apparatus includes a bioreactor, a feed container, a heating means such as a heat exchanger and an industrial facility with a heat waste source.

### CROSS REFERENCE TO RELATED APPLICATION

This application claims priority under 35 U.S.C. §119(e) to U.S. Provisional Patent Application Ser. No. 60/686,008, filed May 31, 2005, entitled “HYDROGEN PRODUCING APPARATUS UTILIZING EXCESS HEAT FROM A INDUSTRIAL FACILITY”

### FIELD OF THE INVENTION

The present invention relates generally to an apparatus for concentrated production of hydrogen from hydrogen producing microorganism cultures. More particularly, the invention relates to an apparatus that synergistically combines a hydrogen production system with an industrial facility, wherein the industrial facility may be unrelated to the production of hydrogen apart from the claimed apparatus. The hydrogen production system uses heat or heat waste that is produced during typical usage of the industrial facility, thereby reducing energy costs of the hydrogen production system and conserving energy from the facility. The industrial facility may also produce organic waste products that are utilized as a hydrogen microorganism organic feed material in the apparatus.

### BACKGROUND OF THE INVENTION

The production of hydrogen is an increasingly common and important procedure in the world today. Production of hydrogen in the U.S. alone currently amounts to about 3 billion cubic feet per year, with output likely to increase. Uses for the produced hydrogen are varied, ranging from uses in welding to production of hydrochloric acid. An increasingly important use of hydrogen relates to the production of alternative fuels for machinery such as motor vehicles. Successful use of hydrogen as an alternative fuel can provide substantial benefits to the world at large. This is important not only in that the hydrogen can be formed without dependence on the location of specific oils or other ground resources, but in that burning of hydrogen for fuel is atmospherically clean. Essentially, no carbon dioxide or greenhouse gasses are produced during the burning. Thus, production of hydrogen is an environmentally desirable goal.

Creation of hydrogen from certain methods and apparatuses are generally known. For example, electrolysis, which generally involves the use of electricity to decompose water into hydrogen and oxygen, is a commonly used process. Significant energy, however, is required to produce the needed electricity to perform the process. Similarly, steam reforming is another expensive method requiring fossil fuels as an energy source. As could be readily understood, the environmental benefits of producing hydrogen are at least partially offset when using a process that uses pollution-causing fuels as an energy source for the production of hydrogen.

New methods of hydrogen generation are therefore needed. One possible method is to create hydrogen in a biological system by converting organic matter into hydrogen gas. The creation of a biologically produced gas that is substantially hydrogen can theoretically be achieved in a bioreactor, wherein hydrogen producing microorganisms and an organic feed material are held in an environment favorable to hydrogen production. Substantial and useful creation of hydrogen gas from micro-organisms, however, is problematic. The primary obstacle to sustained production of useful quantities of hydrogen by micro-organisms has been the eventual stoppage of hydrogen production generally coinciding with the appearance of methane. This occurs when methanogenic microorganisms in the bioreactor environment convert hydrogen to methane. This process occurs naturally in anaerobic environments such as marshes, swamps, and pond sediments. As the appearance of methanogens in a biological system has previously been largely inevitable, continuous production of hydrogen from hydrogen producing micro-organisms has been unsuccessful in the past.

Microbiologists have for many years known of organisms which generate hydrogen as a metabolic by-product. Two reviews of this body of knowledge are Kosaric and Lyng (1988) and Nandi and Sengupta (1998). Among the various organisms mentioned, the heterotrophic facultative anaerobes are of interest; particularly those in the group known as the enteric microorganisms. Within this group are the mixed-acid fermenters, whose most well known member is Escherichia coli. While fermenting glucose, these micro-organisms split the glucose molecule forming two moles of pyruvate (Equation 1); an acetyl group is stripped from each pyruvate fragment leaving formic acid (Equation 2), which is then cleaved into equal amounts of carbon dioxide and hydrogen as shown in simplified form below (Equation 3).
Glucose→2 Pyruvate (1)
2 Pyruvate+2 Coenzyme A→2 Acetyl-CoA+2 HCOOH (2)
2 HCOOH→2 H₂+2 CO₂ (3)

Thus, during this process, one mole of glucose produces two moles of hydrogen gas. Also produced during the process are acetic and lactic acids, and minor amounts of succinic acid and ethanol. Other enteric microorganisms (the 2, 3 butanediol fermenters) use a different enzyme pathway which causes additional CO₂ generation resulting in a 6:1 ratio of carbon dioxide to hydrogen production (Madigan et al., 1997). After this process, the hydrogen is typically converted into methane by methanogens.

There are many sources of waste organic matter which could serve as a substrate for this microbial process. One such material would be organic-rich industrial wastewaters, particularly sugar-rich waters, such as fruit and vegetable processing wastes. Other sources include agricultural residues and other organic wastes such as sewage and manures.

Like some hydrogen producing apparatuses and processes, many industrial facilities and processes have flowing streams of liquids, solids, or gases that contain heat or waste products that must be exhausted to the environment or removed in some way for safety or proper function of the process. The excess heat from these industrial facilities can be environmentally harmful and/or wasteful. In some occasions, the industrial facility or process will recycle excess heat back into its apparatus or process with heat exchangers or other process streams. Other heat, however, is not recycled due to lack of need of the facility or lack of suitability of the heat. Any heat which is not recycled into the facility is typically referred to as heat waste. Most often heat waste is simply discharged to the environment, either directly as an exhaust stream, or indirectly via a cooling medium such as cooling water.

New types of hydrogen generation are therefore needed that produce substantial and useful levels of hydrogen in an inexpensive, environmentally sound apparatus that additionally reduces the amount of heat waste produced in a typical industrial facility.

### SUMMARY OF THE INVENTION

Therefore, it is an object of the present invention to create a biological system in a bioreactor wherein hydrogen is produced by hydrogen producing microorganisms by utilizing heat or heat waste from an industrial facility to deactivate or kill methanogens.

It is a further object of the invention to provide an apparatus for producing hydrogen from an organic feed material having a bioreactor adapted receive therein the organic feed material to produce the hydrogen from microorganisms metabolizing the organic feed material, means for heating the organic feed material before it is introduced into the bioreactor, wherein methanogens in the organic feed material are substantially killed or deactivated; and means for removing the hydrogen from the bioreactor.

It is a further object of the invention to provide an apparatus that includes a bioreactor readily combinable and proximate with wide variety of industrial facilities of differing products, the bioreactor utilizing heat and organic waste from an industrial facility to create hydrogen, wherein the hydrogen is not substantially converted to methane subsequent to production.

It is a further object of the invention to heat the organic feed material prior to entry into the bioreactor, wherein heating is achieved in any one or a multiplicity of upstream containers or passages, such that heating the organic feed material at temperatures of about 60 to 100° C. kills or deactivates methanogens while leaving hydrogen producing microorganisms intact. Heating is preferably achieved by using a heat exchanger to capture excess heat from an industrial facility.

It is a further object of the invention to include other means to further treat the organic feed material, such as aerating the organic feed material, diluting the organic feed material, inoculating the organic feed material with additional hydrogen producing microorganisms, or adding other chemical supplements. Treatments may occur in the bioreactor or further upstream the bioreactor.

It is a further object to use an organic feed material that is equivalent to or derived from wastewater exhausted by the same industrial facility that is exhausting heat waste used to heat the organic feed material.

It is a further object of the invention to provide an industrial facility that produces byproducts of an organic feed material and heat, the industrial facility bring adapted to convert the organic feed material into hydrogen, the industrial facility comprising a bioreactor adapted to receive the organic feed material and to produce the hydrogen from microorganisms metabolizing the organic feed material, means for heating the organic feed material before it is introduced into the bioreactor, wherein methanogens in the organic feed material are substantially killed or deactivated, and means for removing the hydrogen from the bioreactor.

These and other objects of the present invention will become more readily apparent from the following detailed description and appended claims.

### BRIEF DESCRIPTION OF DRAWINGS

FIG. 1 is a plan view of the hydrogen production system.

FIG. 2 is a side view of one embodiment of the bioreactor.

FIG. 3 is a plan view the bioreactor.

FIG. 4 is a plan view of coated substrates.

FIG. 5 is a top plan view of a system layout in a housing unit.

### DETAILED DESCRIPTION OF PREFERRED EMBODIMENTS

As used herein, the term “microorganisms” include bacteria and substantially microscopic cellular organisms.

As used herein, the term “hydrogen producing microorganisms” includes microorganisms that metabolize an organic substrate in one or a series of reactions that ultimately form hydrogen as one of the end products.

As used herein, the term “methanogens” refers to microorganisms that metabolize hydrogen in one or a series of reactions that produce methane as one of the end products.

As used herein, the term “replenishable coating” refers to coating that can be replaced or supplemented by the introduction of additional coating.

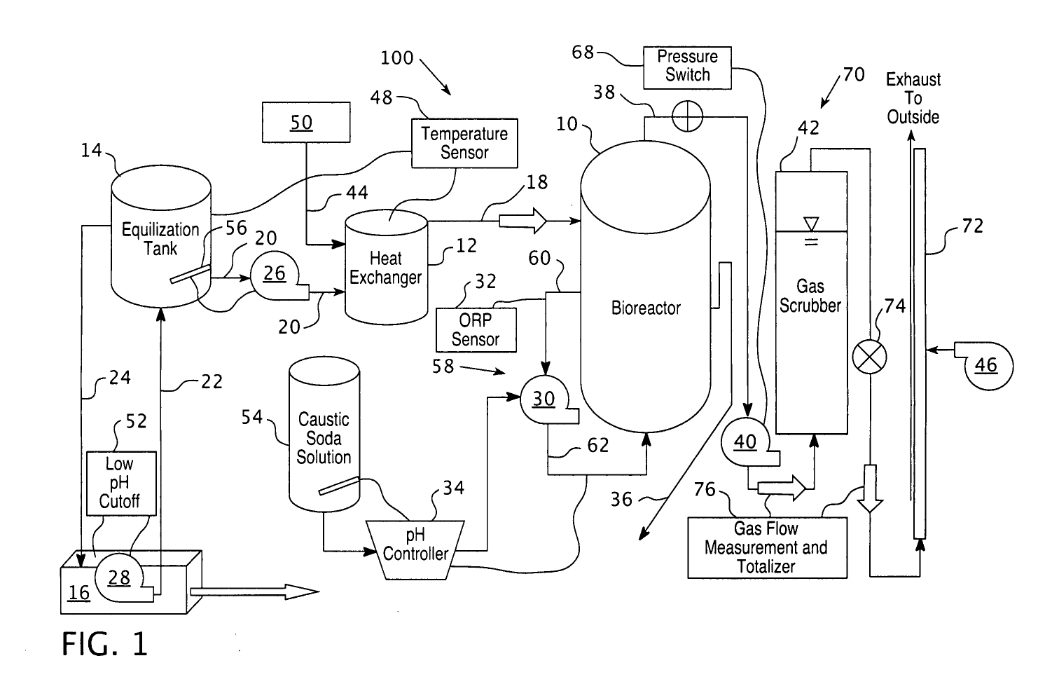

A hydrogen producing system 100 for sustained production of hydrogen in accordance with the present invention is shown in FIG. 1, including industrial facility 50, passage 44, heat exchanger 12 and a multiplicity of containers, wherein the containers include bioreactor 10, heat exchanger 12, equalization tank 14 and reservoir 16. The apparatus enables the production of sustained hydrogen containing gas in bioreactor 10, wherein the produced gas substantially produces a 1:1 ratio of hydrogen to carbon dioxide gas and does not substantially include any methane. The hydrogen containing gas is produced by the metabolism of an organic feed material by hydrogen producing microorganisms. In preferred embodiments, organic feed material is a sugar containing organic feed material. In further preferred embodiments, the organic feed material is industrial wastewater or effluent product that is produced during routine formation of fruit and/or vegetable juices, such as grape juice. In additional embodiments, wastewaters rich not only in sugars but also in protein and fats could be used, such as milk product wastes. The most complex potential source of energy for this process would be sewage-related wastes, such as municipal sewage sludge and animal manures. However, any feed containing organic material is usable in hydrogen producing system 100. Hydrogen producing microorganisms can metabolize the sugars in the organic feed material under the reactions:
Glucose→2 Pyruvate (1)
2 Pyruvate+2 Coenzyme A→2 Acetyl-CoA+2 HCOOH (2)
2 HCOOH→2 H₂+2 CO₂ (3)

During this process, one mole of glucose produces two moles of hydrogen gas and carbon dioxide. In alternate embodiments, other organic feed materials include agricultural residues and other organic wastes such as sewage and manures. Typical hydrogen producing microorganisms are adept at metabolizing the high sugar organic waste into bacterial waste products. The wastewater may be further treated by aerating, diluting the solution with water or other dilutants, adding compounds that can control the pH of the solution or other treatment step. For example, the solution may be supplemented with phosphorus (NaH₂PO₄) or yeast extract.

Organic feed material provides a plentiful feeding ground for hydrogen producing microorganisms and is naturally infested with these microorganisms. While hydrogen producing microorganisms typically occur naturally in an organic feed material, the organic feed material is preferably further inoculated with hydrogen producing microorganisms in an inoculation step. The inoculation may be an initial, one-time addition to bioreactor 10 at the beginning of the hydrogen production process. Further inoculations, however, may be added as desired. The added hydrogen producing microorganisms may include the same types of microorganisms that occur naturally in the organic feed material. In preferred embodiments, the hydrogen producing microorganisms, whether occurring naturally or added in an inoculation step, are preferably microorganisms that thrive in pH levels of about 3.5 to 6.0 and can survive in temperature of 60-100° F. or, more preferably, 60-75°. These hydrogen producing microorganisms include, but are not limited to, Clostridium sporogenes, Bacillus licheniformis and Kleibsiella oxytoca. Hydrogen producing microorganisms can be obtained from a microorganismal culture lab or like source. Other hydrogen producing microorganisms or microorganisms known in the art, however, can be used within the spirit of the invention. The inoculation step can occur in bioreactor 10 or elsewhere in the apparatus, for example, recirculation system 58.

Reservoir 16 is a container known in the art that can contain an organic feed material. The size, shape, and material of reservoir 16 can vary widely within the spirit of the invention. In one embodiment, reservoir 16 is one or a multiplicity of storage tanks that are adaptable to receive, hold and store the organic feed material when not in use, wherein the one or a multiplicity of storage tanks may be mobile. In preferred embodiments, reservoir 16 is a wastewater well that is adaptable to receive and contain wastewater and/or effluent from an industrial facility 50. In further preferred embodiments, reservoir 16 is adaptable to receive and contain wastewater that is effluent from a juice manufacturing industrial facility 50, such that the effluent held in the reservoir is a sugar rich juice sludge.

Organic feed material contained in reservoir 16 can be removed through passage 22 with pump 28. Pump 28 is in operable relation to reservoir 16 such that it aids removal movement of organic feed material 16 into passage 22 at a desired, adjustable flow rate, wherein pump 28 can be any pump known in the art suitable for pumping liquids. In a preferred embodiment, pump 28 is a submersible sump pump. Reservoir 16 may further include a low pH cutoff device 52, such that exiting movement into passage 22 of the organic feed material is ceased if the pH of the organic feed material is outside of a desired range. The pH cutoff device 52 is a device known in the art operably related to reservoir 16 and pump 28. If the monitor detects a pH of a solution in reservoir 16 out of range, the device ceases operation of pump 28. The pH cut off in reservoir 16 is typically greater than the preferred pH of bioreactor 10. In preferred embodiments, the pH cutoff 52 is set between about 7 and 8 pH. In alternate embodiments, particularly when reservoir 16 is not adapted to receive effluent from an industrial facility 50, the pH cutoff device is not used.

Passage 22 provides further entry access into equalization tank 14 or heat exchanger 12. Equalization tank is an optional intermediary container for holding organic feed material between reservoir 16 and heat exchanger 12. Equalization tank 14 provides an intermediary container that can help control the flow rates of organic feed material into heat exchanger 12 by providing a slower flow rate into passage 20 than the flow rate of organic feed material into the equalization tank through passage 22. The equalization tank can be formed of any material suitable for holding and treating the organic feed material. In the present invention, equalization tank 14 is constructed of high density polyethylene materials. Other materials include, but are not limited to, metals or acrylics. Additionally, the size and shape of equalization tank 14 can vary widely within the spirit of the invention depending on throughput and output and location limitations. In preferred embodiments, equalization tank 14 further includes a low level cut-off point device 56. The low-level cut-off point device ceases operation of pump 26 if organic feed material contained in equalization tank 14 falls below a predetermined level. This prevents air from entering passage 20. Organic feed material can be removed through passage 20 or through passage 24. Passage 20 provides removal access from equalization tank 14 and entry access into heat exchanger 12. Passage 24 provides removal access from equalization tank 14 of solution back to reservoir 16. Passage 24 provides a removal system for excess organic feed material that exceeds the cut-off point of equalization tank 14. Both passage 20 and passage 24 may further be operably related to pumps to facilitate movement of the organic feed material. In alternate embodiments, equalization tank 14 is not used and organic feed material moves directly from reservoir 16 to heat exchanger 12. In these embodiments, passages connecting reservoir 16 and heat exchanger 12 are arranged accordingly.

The organic feed material is heated prior to conveyance into the bioreactor. The heating can occur anywhere upstream. In one embodiment, the heating is achieved in one or a multiplicity of heat exchangers 12, wherein the organic feed material is heated within the heat exchanger 12. Organic feed solution can be additionally heated at additional or alternate locations in the hydrogen production system. Passage 20 provides entry access to heat exchanger 12, wherein heat exchanger 12 is any apparatus known in the art that can contain and heat contents held within it. Passage 20 is preferably operably related to pump 26. Pump 26 aids the conveyance of solution from equalization tank 14 or reservoir 16 into heat exchanger 12 through passage 20, wherein pump 26 is any pump known in the art suitable for this purpose. In preferred embodiments, pump 26 is an air driven pump for ideal safety reasons. However, motorized pumps are also found to be safe and are likewise usable.

A heating source for system 100 preferably is heat exchanger 12 that uses heat or heat waste from industrial facility 50 to heat the organic feed material, wherein the heat exchanger is a heat exchanger known in the art. The heat waste may be transferred through passage 44.

The heat exchanger can be a liquid phase-liquid phase or gas-phase/liquid phase as dictated by the phase of the heat waste. A typical heat exchanger, for example, is a shell and tube heat exchanger which consists of a series of finned tubes, through which a first fluid runs. A second fluid runs over the finned tubes to be heated or cooled. Another type of heat exchanger is a plate heat exhanger, which directs flow through baffles so that fluids to be ehated and cooled are separated by plates with very large surface area. The heat exchanger 12 heats the organic feed material.

Heat is captured from industrial facility 50 and used to partially or fully heat the organic feed material, wherein industrial facility 50 includes a heat waste source. There is great diversity among these types of industrial facilities 50 in terms of types and order of processing steps, and there is even wide variance between industrial facilities that produce the same product. In a preferred embodiment, the industrial facility 50 is a juice or food manufacturing facility. A typical industrial juice facility involves most of the following basic processes: sorting, washing, extracting, pressing, straining, pasteurizing, heat sterilization, boiling, drying, evaporating, filling, sealing, and labeling. Further, prior to being filled by juice or food, a can, glass or bottle container may be cleaned by hot water, steam or air blast. Further, containers may be exhausted to remove air such that pressure inside the container is less than atmospheric. Heat exchanger 12 receives heat waste from the industrial facility 50 through passage 44 at these or any location where heat waste is produced to elevate the temperature of organic feed material to about 60 to 100° C. Passage 44 may further be associated with a pump device to control flow rates. After exiting heat exchanger 12, heat waste originally conveyed through passage 44 may be discarded through an effluent pipe (not pictured) or recycled back into the secondary hydrogen production apparatus. These typically will be the drying, boiling, pasteurizing or heat sterilization processing steps.

In preferred embodiments, industrial facility 50 also provides waste products that are organic feed materials. For example, if industrial facility 50 is juice manufacturing facility and the organic feed material is a waste product from a juice manufacturing facility, then the invention therein provides an apparatus that combines a hydrogen bioreactor 10 with industrial facility 50 such that industrial facility 50 provides both the organic feed material and the heat waste source to heat the organic feed material for hydrogen production.

To allow hydrogen producing microorganisms within the bioreactor 10 to metabolize the organic feed material and produce hydrogen without subsequent conversion of the hydrogen to methane by methanogens, methanogens contained within the organic feed material are substantially killed or deactivated. In preferred embodiments, the methanogens are substantially killed or deactivated prior to entry into the bioreactor. In further preferred embodiments, methanogens contained within the organic feed material are substantially killed or deactivated by being heated under elevated temperatures in heat exchanger 12. Methanogens are substantially killed or deactivated by elevated temperatures. Methanogens are generally deactivated when heated to temperatures of about 60-75° C. for a period of at least 15 minutes. Additionally, methanogens are generally damaged or killed when heated to temperatures above about 90° C. for a period of at least 15 minutes. Heat exchanger 12 enables heating of the organic feed material to temperature of about 60-100° C. in order to substantially deactivate or kill the methanogens while leaving any hydrogen producing microorganisms substantially functional. This effectively pasteurizes or sterilizes the contents of the organic feed material from active methanogens while leaving the hydrogen producing microorganisms intact, thus allowing the produced biogas to include hydrogen without subsequent conversion to methane. The size, shape and numbers of heat exchangers 12 can vary widely within the spirit of the invention depending on throughput and output required and location limitations. In preferred embodiments, retention time in heat exchanger 12 is at least 20 minutes. Retention time marks the average time any particular part of organic feed material is retained in heat exchanger 12.

At least one temperature sensor 48 monitors a temperature indicative of the organic feed material temperature, preferably the temperature levels of equalization tank 14 and/or heat exchanger 12. In preferred embodiments, an electronic controller is provided having at least one microprocessor adapted to process signals from one or a plurality of devices providing organic feed material parameter information, wherein the electronic controller is operably related to the at least one actuatable terminal and is arranged to control the operation of and to controllably heat the heat exchanger 12 and/or any contents therein. The electronic controller is located or coupled to heat exchanger 12 or equalization tank 14, or can alternatively be at a third or remote location. In alternate embodiments, the controller for controlling the temperature of heat exchanger 12 is not operably related to temperature sensor 48.

Passage 18 connects heat exchanger 12 with bioreactor 10. Organic feed material is conveyed into the bioreactor through transport passage 18 at a desired flow rate. System 100 is a continuous flow system with organic feed material in constant motion between containers such as reservoir 16, heat exchanger 12, bioreactor 10, equalization tank 14 if applicable, and so forth. Flow rates between the container can vary depending on retention time desired in any particular container. For example, in preferred embodiments, retention time in bioreactor 10 is between about 6 and 12 hours. To meet this retention time, the flow rate of passage 18 and effluent passage 36 are adjustable as known in the art so that organic feed material, on average, stays in bioreactor 10 for this period of time.

The organic feed material is conveyed through passage 18 having a first and second end, wherein passage 18 provides entry access to the bioreactor at a first end of passage 18 and providing removal access to the heat exchanger 12 at a second end of passage 18. Any type of passage known in the art can be used, such as a pipe or flexible tube. The transport passage may abut or extend within the bioreactor and/or the heat exchanger 12. Passage 18 can generally provide access to bioreactor 10 at any location along the bioreactor. However, in preferred embodiments, passage 18 provides access at an upper portion of bioreactor 10.

Bioreactor 10 provides an anaerobic environment conducive for hydrogen producing microorganisms to grow, metabolize organic feed material, and produce hydrogen. While the bioreactor is beneficial to the growth of hydrogen producing microorganisms and the corresponding metabolism of organic feed material by the hydrogen producing microorganisms, it is preferably restrictive to the proliferation of unwanted microorganisms such as methanogens, wherein methanogens are microorganisms that metabolize carbon dioxide and hydrogen to produce methane and water. Methanogens are obviously unwanted as they metabolize hydrogen. If methanogens were to exist in substantial quantities in bioreactor 10, hydrogen produced by the hydrogen producing bacteria will subsequently be converted to methane, reducing the percentage of hydrogen in the produced gas.

Bioreactor 10 can be any receptacle known in the art for holding an organic feed material. Bioreactor 10 is substantially airtight, providing an anaerobic environment. Bioreactor 10 itself may contain several openings. However, these openings are covered with substantially airtight coverings or connections, such as passage 18, thereby keeping the environment in bioreactor 10 substantially anaerobic. Generally, the receptacle will be a limiting factor for material that can be produced. The larger the receptacle, the more hydrogen producing bacteria containing organic feed material, and, by extension, hydrogen, can be produced. Therefore, the size and shape of the bioreactor can vary widely within the sprit of the invention depending on throughput and output and location limitations.

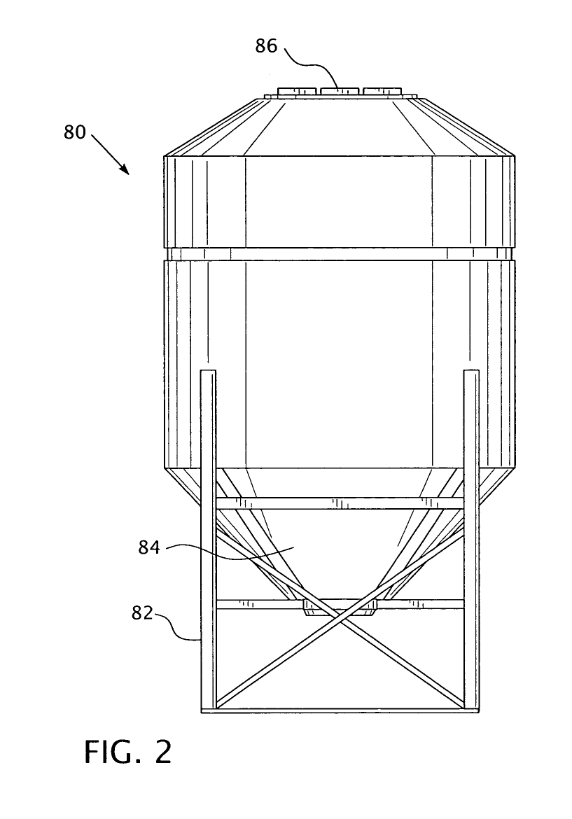

A preferred embodiment of a bioreactor is shown in FIG. 2. Bioreactor 80 can be formed of any material suitable for holding an organic feed material and that can further create an airtight, anaerobic environment. In the present invention, bioreactor 10 is constructed of high density polyethylene materials. Other materials, including but not limited to metals or plastics can similarly be used. A generally silo-shaped bioreactor 80 has about a 300 gallon capacity with a generally conical bottom 84. Stand 82 is adapted to hold cone bottom 84 and thereby hold bioreactor 80 in an upright position. The bioreactor 80 preferably includes one or a multiplicity of openings that provide a passage for supplying or removing contents from within the bioreactor. The openings may further contain coverings known in the art that cover and uncover the openings as desired. For example, bioreactor 80 preferably includes a central opening covered by lid 86. In alternate embodiments of the invention, the capacity of bioreactor 80 can be readily scaled upward or downward depending on needs or space limitations.

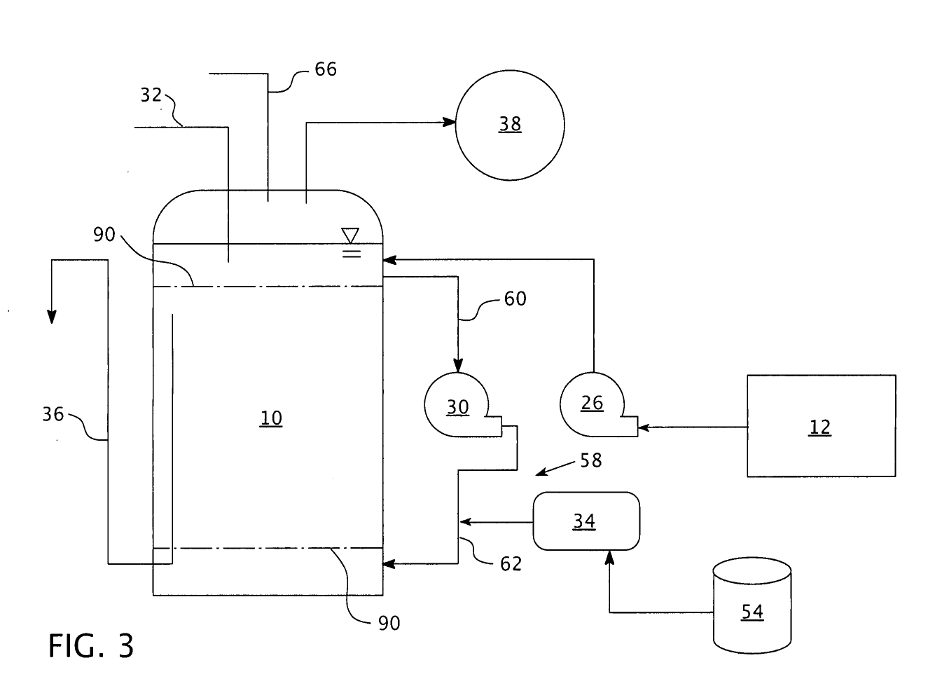

To maintain the solution volume level at a generally constant level, the bioreactor preferably provides a system to remove excess solution, as shown in FIGS. 1 and 3. In the present embodiment, the bioreactor includes effluent passage 36 having an open first and second end that provides a passage from inside bioreactor 10 to outside the bioreactor. The first end of effluent passage 36 may abut bioreactor 10 or extend into the interior of bioreactor 10. If effluent passage 36 extends into the interior of passage 10, the effluent passage preferably extends upwards to generally upper portion of bioreactor 10. When bioreactor 10 is filled with organic feed material, the open first end of the effluent passage allows an excess organic feed material to be received by effluent passage 36. Effluent passage 36 preferably extends from bioreactor 10 into a suitable location for effluent, such as a sewer or effluent container, wherein the excess organic feed material will be deposited through the open second end.

Bioreactor 10 preferably contains one or a multiplicity of substrates 90 for providing surface area for attachment and growth of bacterial biofilms. Sizes and shapes of the one or a multiplicity of substrates 90 can vary widely, including but not limited to flat surfaces, pipes, rods, beads, slats, tubes, slides, screens, honeycombs, spheres, object with latticework, or other objects with holes bored through the surface. Numerous substrates can be used, for example, hundreds, as needed. The more successful the biofilm growth on the substrates, the more fixed state hydrogen production will be achieved. The fixed nature of the hydrogen producing microorganisms provide the sustain production of hydrogen in the bioreactor.

Substrates 90 preferably are substantially free of interior spaces that potentially fill with gas. In the present embodiment, the bioreactor comprises about 100-300 pieces of 1″ plastic media to provide surface area for attachment of the bacterial biofilm. In one embodiment, substrates 90 are Flexiring™ Random Packing (Koch-Glitsch.) Some substrates 90 may be retained below the liquid surface by a retaining device, for example, a perforated acrylic plate. In this embodiment, substrates 90 have buoyancy, and float on the organic feed material. When a recirculation system is operably, the buoyant substrates stay at the same general horizontal level while the organic feed material circulates, whereby providing greater access to the organic feed material by hydrogen producing microorganism- and nonparaffinophilic microorganism-containing biofilm growing on the substrates.

In preferred embodiments, a recirculation system 58 is provided in operable relation to bioreactor 10. Recirculation system 58 enables circulation of organic feed material contained within bioreactor 10 by removing organic feed material at one location in bioreactor 10 and reintroduces the removed organic feed material at a separate location in bioreactor 10, thereby creating a directional flow in the bioreactor. The directional flow aids the microorganisms within the organic feed material in finding food sources and substrates on which to grown biofilms. As could be readily understood, removing organic feed material from a lower region of bioreactor 10 and reintroducing it at an upper region of bioreactor 10 would create a downward flow in bioreactor 10. Removing organic feed material from an upper region of bioreactor 10 and reintroducing it at a lower region would create an up-flow in bioreactor 10.

In preferred embodiments, as shown in FIG. 1, recirculation system 58 is arranged to produce an up-flow of any solution contained in bioreactor 10. Passage 60 provides removal access at a higher point than passage 62 provides entry access. Pump 30 facilitates movement from bioreactor 10 into passage 60, from passage 60 into passage 62, and from passage 62 back into bioreactor 10, creating up-flow movement in bioreactor 10. Pump 30 can be any pump known in the art for pumping organic feed material. In preferred embodiments, pump 30 is an air driven centrifugal pump. Other arrangements can be used, however, while maintaining the spirit of the invention. For example, a pump could be operably related to a single passage that extends from one located of the bioreactor to another.

Bioreactor 10 may optionally be operably related to one or a multiplicity of treatment apparatuses for treating organic feed material contained within bioreactor 10 for the purpose of making the organic feed material more conducive to proliferation of hydrogen producing microorganisms. The one or a multiplicity of treatment apparatuses perform operations that include, but are to limited to, aerating the organic feed material, diluting the organic feed material with water or other dilutant, controlling the pH of the organic feed material, and adding additional chemical compounds to the organic feed material. The apparatus coupled to the bioreactor can be any apparatuses known in the art for incorporating these treatments. For example, in one embodiment, a dilution apparatus is a tank having a passage providing controllable entry access of a dilutant, such as water, into bioreactor 10. An aerating apparatus is an apparatus known in the art that provides a flow of gas into bioreactor 10, wherein the gas is typically air. A pH control apparatus is an apparatus known in the art for controlling a pH of a solution. Additionally chemical compounds added by treatment apparatuses include anti-fungal agents, phosphorous supplements, yeast extract or hydrogen producing microorganism inoculation. In other embodiments, the one or a multiplicity of treatment apparatuses may be operably related to other parts of the bioreactor system. For example, in one example, the treatment apparatuses are operably related to equalization tank 14 or recirculation system 58. In still other embodiments, multiple treatment apparatus of the same type may be located at various points in the bioreactor system to provide treatments at desired locations.

Certain hydrogen producing bacteria proliferate in pH conditions that are not favorable to methanogens, for example, Kleibsiella oxytoca. Keeping organic feed material contained within bioreactor 10 within this favorable pH range is conducive to hydrogen production. In preferred embodiments, pH controller 34 monitors the pH level of contents contained within bioreactor 10. In preferred embodiments, the pH of the organic feed material in bioreactor 10 is maintained at about 3.5 to 6.0 pH, most preferably at about 4.5 to 5.5 pH, as shown in Table 2. In further preferred embodiments, pH controller 34 controllably monitors the pH level of the organic feed material and adjustably controls the pH of the solution if the solution falls out of or is in danger of falling out of the desired range. As shown in FIG. 1, pH controller 34 monitors the pH level of contents contained in passage 62, such as organic feed material, with pH sensor 64. As could readily be understood, pH controller 34 can be operably related to any additional or alternative location that potentially holds organic feed material, for example, passage 60, passage 62 or bioreactor 10 as shown in FIG. 3.

If the pH of the organic feed material falls out of a desired range, the pH is preferably adjusted back into the desired range. Precise control of a pH level is necessary to provide an environment that enables at least some hydrogen producing bacteria to function while similarly providing an environment unfavorable to methanogens. This enables the novel concept of allowing microorganism reactions to create hydrogen without subsequently being overrun by methanogens that convert the hydrogen to methane. Control of pH of the organic feed material in the bioreactor can be achieved by any means known in the art. In one embodiment, a pH controller 34 monitors the pH and can add a pH control solution from container 54 in an automated manner if the pH of the bioreactor solution moves out of a desired range. In a preferred embodiment, the pH monitor controls the bioreactor solution's pH through automated addition of a sodium or potassium hydroxide solution. One such apparatus for achieving this is an Etatron DLX pH monitoring device. Preferred ranges of pH for the bioreactor solution is between about 3.5 and 6.0, with a more preferred range between about 4.0 and 5.5 pH.

The hydrogen producing reactions of hydrogen producing bacteria metabolizing organic feed material in bioreactor 10 can further be monitored by oxidation-reduction potential (ORP) sensor 32. ORP sensor 32 monitors redox potential of organic feed material contained within bioreactor 10. Once ORP drops below about −200 mV, gas production commences. Subsequently while operating in a continuous flow mode, the ORP was typically in the range of −300 to −450 mV.

In one embodiment, the wastewater is a grape juice solution prepared using Welch's Concord Grape Juice™ diluted in tap water at approximately 32 mL of juice per Liter. The solution uses chlorine-free tap water or is aerated previously for 24 hours to substantially remove chlorine. Due to the acidity of the juice, the pH of the organic feed material is typically around 4.0. The constitutional make-up of the grape juice solution is shown in Table 1.

TABLE 1
Composition of concord grape juice. Source:
Welch's Company, personal comm., 2005.

Concentration (unit indicated)

| Constituent | Mean | Range |
|---|---|---|
| Carbohydrates¹ | | 15-18% |
| glucose | 6.2% | 5-8% |
| fructose | 5.5% | 5-8% |
| sucrose | 1.8% | 0.2-2.3% |
| maltose | 1.9% | 0-2.2% |
| sorbitol | 0.1% | 0-0.2% |
| Organic Acids¹ | | 0.5-1.7% |
| Tartaric acid | 0.84% | 0.4-1.35% |
| Malic acid | 0.86% | 0.17-1.54% |
| Citric acid | 0.044% | 0.03-0.12% |
| Minerals¹ | | |

| | | |
|---|---|---|
| Calcium | 17-34 | mg/L |
| Iron | 0.4-0.8 | mg/L |
| Magnesium | 6.3-11.2 | mg/L |
| Phosphorous | 21-28 | mg/L |
| Potassium | 175-260 | mg/L |
| Sodium | 1-5 | mg/L |
| Copper | 0.10-0.15 | mg/L |
| Manganese | 0.04-0.12 | mg/L |
| Vitamins¹ | | |
| Vitamin C | 4 | mg/L |
| Thiamine | 0.06 | mg/L |
| Riboflavin | 0.04 | mg/L |
| Niacin | 0.2 | mg/L |
| Vitamin A | 80 | I.U. |

| | |
|---|---|
| pH | 3.0-3.5 |
| Total solids | 18.5% |

¹additional trace constituents in these categories may be present.

Bioreactor 10 further preferably includes an overflow cut-off switch 66 to turn off pump 26 if the solution exceeds or falls below a certain level in the bioreactor.

Bioreactor 10 further includes an apparatus for capturing the hydrogen containing gas produced by the hydrogen producing bacteria. Capture and cleaning methods can vary widely within the spirit of the invention. In the present embodiment, as shown in FIG. 1, gas is removed from bioreactor 10 through passage 38, wherein passage 38 is any passage known in the art suitable for conveying a gaseous product. Pump 40 is operably related to passage 38 to aid the removal of gas from bioreactor 10 while maintaining a slight negative pressure in the bioreactor. In preferred embodiments, pump 40 is an air driven pump. The gas is conveyed to gas scrubber 42, where hydrogen is separated from carbon dioxide. Other apparatuses for separating hydrogen from carbon dioxide may likewise be used. The volume of collected gas can be measured by water displacement before and after scrubbing with concentrated NaOH. Samples of scrubbed and dried gas may be analyzed for hydrogen and methane by gas chromatography with a thermal conductivity detector (TCD) and/or with a flame ionization detector (FID). Both hydrogen and methane respond in the TCD, but the response to methane is improved in the FID (hydrogen is not detected by an FID, which uses hydrogen as a fuel for the flame).

Exhaust system 70 exhausts gas. Any exhaust system known in the art can be used. In a preferred embodiment, as shown in FIG. 1, exhaust system includes exhaust passage 72, backflow preventing device 74, gas flow measurement and totalizer 76, and air blower 46.

The organic feed material may be further inoculated in an initial inoculation step with one or a multiplicity of hydrogen producing bacteria, such as Clostridizim sporogenes, Bacillus licheniformis and Kleibsiella oxytoca, while contained in bioreactor 10. These hydrogen producing bacteria are obtained from a bacterial culture lab or like source. Alternatively, the hydrogen producing bacteria that occur naturally in the waste solution can be used without inoculating the solution. In further alternative embodiments, additional inoculations can occur in bioreactor 10 or other locations of the apparatus, for example, heat exchanger 12, equalization tank 14 and reservoir 16.

In the present embodiment, the preferred hydrogen producing bacteria is Kleibsiella oxytoca, a facultative enteric bacterium capable of hydrogen generation. Kleibsiella oxytoca produces a substantially 1:1 ratio of hydrogen to carbon dioxide through organic feed material metabolization, not including impurities. The source of both the Kleibsiella oxytoca may be obtained from a source such yeast extract. In one embodiment, the continuous input of seed organisms from the yeast extract in the waste solution results in a culture of Kleibsiella oxytoca in the bioreactor solution. Alternatively, the bioreactor may be directly inoculated with Kleibsiella oxytoca. In one embodiment, the inoculum for the bioreactor is a 48 h culture in nutrient broth added to diluted grape juice and the bioreactor was operated in batch mode until gas production commenced.

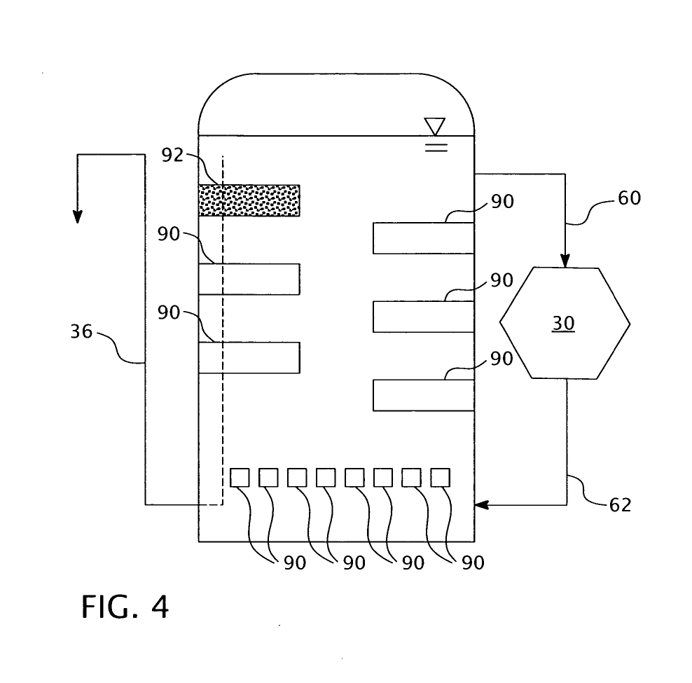

In further embodiments, a carbon-based baiting material is provided within bioreactor 10 as shown FIG. 4. In this embodiment, the apparatus further includes a carbon-based baiting material 92, wherein the carbon based material is preferably coated on the one or a multiplicity of substrates 90 within bioreactor 10. The coating baits nonparaffinophilic microorganisms contained in the organic feed material, which then grow thereon.

Carbon based baiting material 92 is preferably a gelatinous matrix having at least one carbon compound. In one embodiment, the gelatinous matrix is agar based. In this embodiment, the gelatinous matrix is prepared by placing agar and a carbon compound into distilled water, wherein the agar is a gelatinous mix, and wherein any other gelatinous mix known in the art can be used in place of or in addition to agar within the spirit of the invention.

The carbon compound used with the gelatinous mix to form the gelatinous matrix can vary widely within the spirit of the invention. The carbon source is preferably selected from the group consisting of: glucose, fructose, glycerol, mannitol, asparagines, casein, 1-arabinose, cellobiose, dextrose, d-galactose, inositol, lactose, levulose, maltose, d-mannose, melibiose, raffinose, sucrose, d-sorbintol and d-xylose or any combination thereof. Other carbon compounds known in the art, however, can be used within the spirit of the invention.

Generally, the matrix is formed by adding a ratio of three grams of carbon compound and two grams of agar per 100 mL of distilled water. This ratio can be used to form any amount of a mixture up to or down to any scale desired. Once the correct ratio of carbon compound, agar and water are mixed, the mixture is boiled and steam sterilized to form a molten gelatinous matrix. The gelatinous matrix is kept warm within a container such that the mixture remains molten. In one embodiment, the gelatinous matrix is held within a holding container in proximity to substrates 90 until needed to coat the subsrates.

Substrates 90 are coated. The one or a multiplicity of substrates can be any object, shape or material with a hollow or partially hollow interior, wherein the substrate further includes holes that connect the hollow or partially hollow interior to the surface of the substrate. The substrate must also have the ability to withstand heat up to about 100° C. General representative objects and shapes include pipes, rods, beads, slats, tubes, slides, screens, honeycombs, spheres, objects with latticework, or other objects with holes or passages bored through the surface.

In one embodiment, the one or a multiplicity of substrates 90 are generally inserted into the bioreactor through corresponding slots, such that the substrates can be added or removed from the bioreactor without otherwise opening the bioreactor. In alternate embodiments, the substrates are affixed to an interior surface of the bioreactor.

The substrate is coated by carbon based coating material 92. The substrate can be coated by hand, by machine or by any means known in the art. In one embodiment, the carbon based coating material 92 may be coated directly onto the substrate. In alternative embodiments, however, an adhesive layer may be located between the carbon based coating material 92 and the substrate, the adhesive being any adhesive known in the art for holding carbon based compounds. In a preferred embodiment, the adhesive includes a plurality of gel beads, wherein carbon based coating material 92 is affixed to the gel beads ionically or by affinity.

In additional embodiments, coating material 92 is conveyed from the container holding carbon based coating material 92 into a hollow or partially hollow interior channel of the substrate. The gelatinous matrix is conveyed into the channel with a conveying device, preferably a pump. The conveying device can be any pumping means known in the art, including hand or machine. The carbon based coating material 92 permeates from the channel of the substrate to the exterior through the holes, coating the substrate surface. The carbon based coating material 92 on the substrate can be continually replenished at any tine by conveying more gelatinous matrix into the interior of the substrate. The flow of carbon based coating material 92 can be regulated by the conveying device such that the substrate is coated and/or replenished at any speed or rate desired. Further, the entire substrate need not be covered by the carbon based coating material 92, although preferably the majority of the substrate is covered at any moment in time.

In further embodiments, the invention provides a system for producing hydrogen and isolating microorganisms having anaerobic bioreactor for holding organic feed material, one or a multiplicity of substrates contained within the bioreactor, the one or a multiplicity of substrates having a coating disposed thereon for hosting the growth of biofilm, wherein the coating is a replenishable coating from a coating source outside the bioreactor. The coating is contained in a coating container or other container proximate the bioreactor. The system further contains a passage connecting the coating container and the interior channel of one or a multiplicity of substrates. Coating is pumped from the coating container through the passage and into the channel, where the coating permeates from the channel through a permeable or semi-permeable surface of the substrates. As the coating permeates to the surface, it replenishes, i.e., supplements or replaces, coatings already present on the substrates. Alternatively, if no coating is present, the coating permeates to provide an initial coating on the substrates. By replenishing coating, the system has a continuous supply of bait and feeding material for nonparaffinophilic microorganisms. The nonparaffinophilic microorganisms for biofilm on the coated substrates and are thereby isolated on the substrates.

In further embodiments, the one or a multiplicity of substrates are replaceably insertable through openings in the bioreactor. The insertions maintain the anaerobic environment of the bioreactor.

The substrate provides an environment for the development and multiplication of nonparaffinophilic microorganisms in the bioreactor, such as hydrogen producing microorganisms. This is advantageous as substrates enable microorganisms to obtain more nutrients and expend less energy than a similar microorganism floating loosely in organic feed material.

The microorganisms, baited by the carbon based coating material, attach themselves to the substrate, thereby forming a slime layer on the substrate generally referred to as a biofilm. The combination of carbon based coating material 92 on the substrate and the environmental conditions favorable to growth in the organic feed material allows the microorganisms to grow, multiply and form biofilms on the substrate.

In order to increase growth and concentration on the substrate coated with a carbon based baiting means for nonparaffinophilic organisms, the surface area of the substrate can be increased. Increasing the surface area can be achieved by optimizing the surface area of a single substrate within the bioreactor, adding a multiplicity of substrates within the bioreactor, or a combination of both.

The apparatus may further include a coating of alginate within the interior of the bioreactor. The thickness and type of alginate coating can vary within the bioreactor. Thus, the bioreactor may have levels of alginate, i.e., areas of different formulations and amounts of alginate in different locations within the bioreactor.

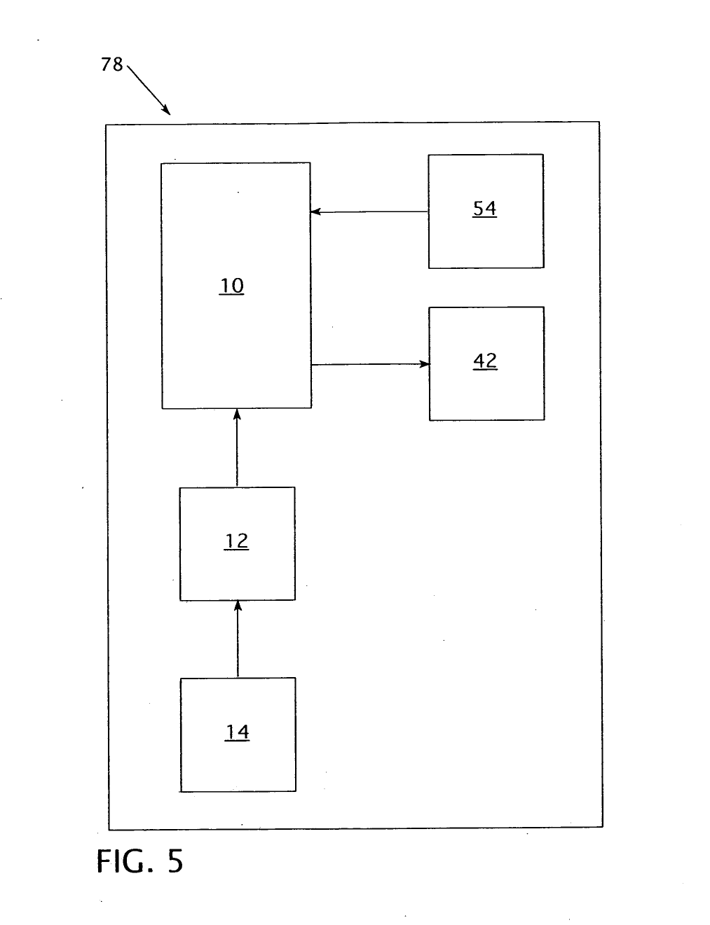

The system may be housed in a single housing unit 68 as shown in FIG. 5. The containers and bioreactors will be filled with liquid and thus will be heavy. For example, if a 300 gallon cone-bottom bioreactor is used. the bioreactor can weigh about 3,000 lbs. The stand preferably has four legs, with a 2″ steel plate tying the legs together. If it is assumed that each leg rests on a 2×2 square, then the loading to the floor at those spots would be 190 lbs/sq inch. The inside vertical clearance is preferably at least 84 inches. For safety reasons, the main light switch for the building will be mounted on the outside next to the entry door and the electrical panel will be mounted on the exterior of the building so that all power to the building could be cut without entering. In this further preferred embodiment, the system is preferably proximate to industrial facility 50.

Hydrogen gas is flammable, but the ignition risk is low, and less than if dealing with gasoline or propane. Hydrogen gas is very light, and will rise and dissipate rapidly. A housing unit is preferably equipped with a vent ridge and eave vents creating natural ventilation. While the LEL (lower explosive limit) for hydrogen is 4%, it is difficult to ignite hydrogen even well above the LEL through electrical switches and motors.

All plumbing connections for the system are water tight, and the gas-side connections are pressure checked. Once the produced gas has been scrubbed of CO2, it will pass through a flow sensor and then be exhausted to the atmosphere through a stand pipe. A blower (as used in boats where gas fumes might be present) will add air to the stand pipe at a rate of more than 500 to 1, thus reducing the hydrogen concentration well below the LEL. As soon as this mixture reaches the top of the pipe, it will be dissipated by the atmosphere.

In case of a leak inside the building, the housing unit preferably includes a hydrogen sensor connected to a relay which will activate an alarm and a ventilation system. The ventilation system is preferably mounted on the outside of the building and will force air through the building and out the roof vents. The hydrogen sensor is preferably set to activate if the hydrogen concentration reaches even 25% of the LEL. The only electrical devices will be a personal computer, low-voltage sensors, electrical outlets and connections, all of which will be mounted on the walls lower than normal. The hydrogen sources will preferably be located high in the room and since hydrogen does not settle.

### EXAMPLE 1

The apparatus combines a bioreactor with a grape juice facility. The organic feed material is a grape juice waste product diluted in tap water at approximately 32 mL of juice per liter. The solution uses chlorine-free tap water or is aerated previously for 24 hours to substantially remove chlorine. The dilution and aeration occur in a treatment container. The organic feed material is then conveyed into the feed container through a passage.

The organic feed material is heated in the feed container to about 65° C. for about 10 minutes to substantially deactivate methanogens. The organic feed material is heated with a heat exchanger with excess heat from the grape juice facility. The organic feed material is conveyed through a passage to the bioreactor wherein it is further inoculated with Kleibsiella oxytoca. The resultant biogases produced by the microorganisms metabolizing the organic feed material include hydrogen without any substantial methane.

### EXAMPLE 2

A multiplicity of reactors were initially operated at pH 4.0 and a flow rate of 2.5 mL min⁻¹, resulting in a hydraulic retention time (HRT) of about 13 h (0.55 d). This is equivalent to a dilution rate of 1.8 d⁻¹. After one week all six reactors were at pH 4.0, the ORP ranged from −300 to −450 mV, total gas production averaged 1.6 L d⁻¹ and hydrogen production averaged 0.8 L d⁻¹. The mean COD of the organic feed material during this period was 4,000 mg L⁻¹ and the mean effluent COD was 2,800 mg L⁻¹, for a reduction of 30%. After one week, the pHs of certain reactors were increased by one half unit per day until the six reactors were established at different pH levels ranging from 4.0 to 6.5. Over the next three weeks at the new pH settings, samples were collected and analyzed each weekday. It was found that the optimum for gas production in this embodiment was pH 5.0 at 1.48 L hydrogen d⁻¹ (Table 2). This was equivalent to about 0.75 volumetric units of hydrogen per unit of reactor volume per day.

TABLE 2
Production of hydrogen in 2-L anaerobic bioreactors as a function of pH.

| | Total | | H2 | H2 per |
|---|---|---|---|---|
| | gas | H2 | L/g | Sugar |
| pH | L/day | L/day | COD | moles/mole |
| 4.0^{a} | 1.61 | 0.82 | 0.23 | 1.81 |
| 4.5^{b} | 2.58 | 1.34 | 0.23 | 1.81 |
| 5.0^{c} | 2.74 | 1.48 | 0.26 | 2.05 |
| 5.5^{d} | 1.66 | 0.92 | 0.24 | 1.89 |
| 6.0^{d} | 2.23 | 1.43 | 0.19 | 1.50 |
| 6.5^{e} | 0.52 | 0.31 | 0.04 | 0.32 |

^{a}mean of 20 data points
^{b}mean of 14 data points
^{c}mean of 11 data points
^{d}mean of 7 data points
^{e}mean of 6 data points

Also shown in Table 2 is the hydrogen production rate per g of COD, which also peaked at pH 5.0 at a value of 0.26 L g⁻¹ COD consumed. To determine the molar production rate, it was assumed that each liter of hydrogen gas contained 0.041 moles, based on the ideal gas law and a temperature of 25° C. Since most of the nutrient value in the grape juice was simple sugars, predominantly glucose and fructose (Table 1 above), it was assumed that the decrease in COD was due to the metabolism of glucose. Based on the theoretical oxygen demand of glucose (1 mole glucose to 6 moles oxygen), one gram of COD is equivalent to 0.9375 g of glucose. Therefore, using those conversions, the molar H₂ production rate as a function of pH ranged from 0.32 to 2.05 moles of H₂ per mole of glucose consumed. As described above, the pathway appropriate to these organisms results in two moles of H₂ per mole of glucose, which was achieved at pH 5.0. The complete data set is provided in Tables 3a and 3b.

Samples of biogas were analyzed several times per week from the beginning of the study, initially using a Perkin Elmer Autosystem GC with TCD, and then later with a Perkin Elmer Clarus 500 GC with TCD in series with an FID. Methane was never detected with the TCD, but trace amounts were detected with the FID (as much as about 0.05%).

Over a ten-day period, the waste solution was mixed with sludge obtained from a methane-producing anaerobic digester at a nearby wastewater treatment plant at a rate of 30 mL of sludge per 20 L of diluted grape juice. There was no observed increase in the concentration of methane during this period. Therefore, it was concluded that the preheating of the feed to 65° C. as described previously was effective in deactivating the organisms contained in the sludge. Hydrogen gas production rate was not affected (data not shown).

Using this example, hydrogen gas is generated using a microbial culture over a sustained period of time. The optimal pH for this culture consuming simple sugars from a simulated fruit juice bottling wastewater was found to be 5.0. Under these conditions, using plastic packing material to retain microbial biomass, a hydraulic residence time of about 0.5 days resulted in the generation of about 0.75 volumetric units of hydrogen gas per unit volume of reactor per day.

Whereas particular embodiments of this invention have been described above for purposes of illustration, it will be evident to those skilled in the art that numerous variations of the details of the present invention may be made without departing from the invention as defined in the appended claims.

TABLE 3a
Bioreactor Operating Data

| | | | | | | | | | | COD | | | | | | | |
|---|---|---|---|---|---|---|---|---|---|---|---|---|---|---|---|---|---|
| | | | GAS | | Liquid Readings | | | | | | Ef- | Re- | | | Performance | | |
| | | col- | Tot | after | Ef- | | | | | | flu- | mov- | | | Total | | |
| | | lec- | vol- | scrub- | flu- | | Net | | | Feed | ent | al | Load- | Con- | gas | H2 | H2 |
| | Reac- | tion | ume | bing | ent | NaOH | Feed | | | (mg/ | (mg/ | (mg/ | ing | sumed | L/ | L/ | L/g |
| Date | tor | hours | (mL) | (mL) | (mL) | (mL) | (mL) | ORP | pH | L) | L) | L) | (g) | (g) | day | day | COD |
| 17-Nov | C | 5.5 | 360 | 200 | 840 | 120 | 720 | −344 | 4.9 | 4,907 | 2,880 | 2,027 | 3.533 | 1.459 | 1.57 | 0.87 | 0.14 |
| 18-Nov | C | 5 | 370 | 200 | 1120 | 70 | 1050 | −328 | 4.9 | 3,680 | 2,480 | 1,200 | 3.864 | 1.260 | 1.78 | 0.96 | 0.16 |
| 29-Nov | C | 4.25 | 415 | 200 | 920 | 50 | 870 | −403 | 4.9 | 5,013 | 3,093 | 1,920 | 4.362 | 1.670 | 2.34 | 1.13 | 0.12 |
| 17-Nov | E | 5.5 | 490 | 270 | 1210 | 115 | 1095 | −352 | 5.0 | 4,907 | 4,747 | 160 | 5.373 | 0.175 | 2.14 | 1.18 | 1.54 |
| 1-Dec | D | 3.5 | 540 | 250 | 710 | 85 | 625 | −395 | 5.0 | 5,173 | 3,573 | 1,600 | 3.233 | 1.000 | 3.70 | 1.71 | 0.25 |
| 17-Nov | F | 5.5 | 475 | 225 | 1120 | 130 | 990 | −367 | 5.0 | 4,907 | 3,760 | 1,147 | 4.858 | 1.135 | 2.07 | 0.98 | 0.20 |
| 5-Dec | D | 4.5 | 580 | 310 | 710 | 77 | 633 | −423 | 5.0 | 4,267 | 3,573 | 694 | 2.701 | 0.439 | 3.09 | 1.65 | 0.71 |
| 6-Dec | D | 3 | 450 | 240 | 490 | 43 | 447 | −420 | 5.0 | 4,853 | 3,253 | 1,600 | 2.169 | 0.715 | 3.60 | 1.92 | 0.34 |
| 17-Nov | D | 3.5 | 680 | 415 | 580 | 83 | 497 | −326 | 5.0 | 4,907 | 4,213 | 694 | 2.439 | 0.345 | 4.66 | 2.85 | 1.20 |
| 2-Dec | D | 3.75 | 640 | 340 | 830 | 66 | 764 | −412 | 5.0 | 4,587 | 3,787 | 800 | 3.504 | 0.611 | 4.10 | 2.18 | 0.56 |
| 22-Nov | C | 3.75 | 460 | 295 | 800 | 50 | 750 | −349 | 5.0 | 4,107 | 1,280 | 2,827 | 3.080 | 2.120 | 2.94 | 1.89 | 0.14 |
| averages | | 4.34 | 496 | 268 | 848 | 81 | 767 | −374.5 | 5.0 | 4,664 | 3,331 | 1,333 | 3.579 | 1.023 | 2.74 | 1.48 | 0.26 |
| 5-Dec | C | 4.5 | 470 | 250 | 900 | 103 | 797 | −429 | 5.4 | 4,267 | 3,413 | 854 | 3.401 | 0.680 | 2.51 | 1.33 | 0.37 |
| 18-Nov | F | 5 | 90 | 45 | 600 | 55 | 545 | −451 | 5.5 | 3,680 | 3,440 | 240 | 2.006 | 0.131 | 0.43 | 0.22 | 0.34 |
| 21-Nov | D | 4 | 130 | 70 | 830 | 80 | 750 | −454 | 5.5 | 3,493 | 3,360 | 133 | 2.620 | 0.100 | 0.78 | 0.42 | 0.70 |
| 22-Nov | D | 3.75 | 360 | 250 | 766 | 69 | 696 | −461 | 5.5 | 4,107 | 2,880 | 1,227 | 2.858 | 0.854 | 2.30 | 1.60 | 0.29 |
| 29-Nov | D | 4.25 | 100 | 50 | 940 | 100 | 840 | −456 | 5.5 | 5,013 | 3,307 | 1,707 | 4.211 | 1.434 | 0.56 | 0.28 | 0.03 |
| 2-Dec | C | 3.75 | 560 | 290 | 810 | 93 | 717 | −430 | 5.5 | 4,587 | 3,573 | 1,014 | 3.289 | 0.727 | 3.52 | 1.86 | 0.40 |
| 6-Dec | C | 3 | 250 | 130 | 570 | 45 | 525 | −428 | 5.5 | 4,853 | 3,627 | 1,226 | 2.548 | 0.644 | 2.00 | 1.04 | 0.20 |
| averages | | 4.04 | 279 | 155 | 774 | 78 | 696 | −444.1 | 5.5 | 4,286 | 3,371 | 914 | 2.982 | 0.636 | 1.66 | 0.92 | 0.24 |
| 21-Nov | E | 4 | 360 | 250 | 930 | 130 | 800 | −400 | 6.0 | 3,493 | 2,987 | 506 | 2.794 | 0.405 | 2.10 | 1.50 | 0.62 |
| 22-Nov | E | 3.75 | 380 | 280 | 820 | 127 | 693 | −411 | 6.0 | 4,107 | 2,453 | 1,653 | 2.846 | 1.146 | 2.43 | 1.79 | 0.24 |
| 29-Nov | E | 4.25 | 360 | 230 | 870 | 71 | 799 | −467 | 6.0 | 5,013 | 1,973 | 3,040 | 4.006 | 2.429 | 2.03 | 1.30 | 0.09 |
| 1-Dec | E | 3.5 | 420 | 250 | 770 | 127 | 643 | −471 | 6.0 | 5,173 | 2,933 | 2,240 | 3.326 | 1.440 | 2.88 | 1.71 | 0.17 |
| 2-Dec | E | 3.75 | 280 | 170 | 540 | 85 | 455 | −443 | 6.0 | 4,587 | 3,360 | 1,227 | 2.087 | 0.558 | 1.79 | 1.09 | 0.30 |
| 5-Dec | E | 4.5 | 410 | 240 | 930 | 156 | 774 | −487 | 6.0 | 4,267 | 3,253 | 1,014 | 3.303 | 0.785 | 2.19 | 1.28 | 0.31 |
| 6-Dec | E | 3 | 380 | 170 | 660 | 105 | 555 | −490 | 6.0 | 4,853 | 2,293 | 2,560 | 2.693 | 1.421 | 2.24 | 1.36 | 0.12 |
| averages | | 3.82 | 354 | 227 | 789 | 114 | 674 | −453 | 6.0 | 4,499 | 2,750 | 1,749 | 3.033 | 1.179 | 2.23 | 1.43 | 0.19 |
| 29-Nov | F | 4.25 | 90 | 45 | 870 | 150 | 720 | −501 | 6.5 | 5,013 | 1,707 | 3,307 | 3.610 | 2.381 | 0.51 | 0.25 | 0.02 |
| 2-Dec | F | 3.75 | 20 | 0 | 810 | 136 | 674 | −497 | 6.5 | 4,587 | 3,573 | 1,014 | 3.092 | 0.683 | 0.13 | 0.00 | 0.00 |
| 22-Nov | F | 3.75 | 120 | 105 | 790 | 128 | 662 | −477 | 6.5 | 4,107 | 2,240 | 1,867 | 2.719 | 1.236 | 0.77 | 0.67 | 0.08 |
| 5-Dec | F | 4.5 | 10 | 0 | 670 | 121 | 549 | −532 | 6.5 | 4,267 | 2,827 | 1,440 | 2.343 | 0.791 | 0.05 | 0.00 | 0.00 |
| 6-Dec | F | 3 | 60 | 50 | 480 | 90 | 390 | −515 | 6.5 | 4,853 | 2.240 | 2,613 | 1.893 | 1.019 | 0.48 | 0.40 | 0.05 |
| 21-Nov | F | 4 | 200 | 100 | 910 | 150 | 760 | −472 | 6.5 | 3,493 | 2,613 | 880 | 2.655 | 0.669 | 1.20 | 0.60 | 0.15 |
| averages | | 3.88 | 83 | 50 | 755 | 129 | 626 | −499 | 6.5 | 4,387 | 2,533 | 1,853 | 2.745 | 1.160 | 0.52 | 0.31 | 0.04 |

TABLE 3b
Bioreactor Operating Data Continued.

| | | | | | | | | | | COD | | | | | | | |
|---|---|---|---|---|---|---|---|---|---|---|---|---|---|---|---|---|---|
| | | | GAS | | Liquid Readings | | | | | | Ef- | Re- | | | Performance | | |
| | | col- | Total | after | Ef- | | | | | | flu- | mov- | | | Total | | |
| | | lec- | vol- | scrub- | flu- | | Net | | | Feed | ent | al | Load- | Con- | gas | H2 | H2 |
| | Reac- | tion | ume | bing | ent | NaOH | Feed | | | (mg/ | (mg/ | (mg/ | ing | sumed | L/ | L/ | L/g |
| Date | tor | hours | (mL) | (mL) | (mL) | (mL) | (mL) | ORP | pH | L) | L) | L) | (g) | (g) | day | day | COD |
| 14-Nov | A | 5 | 540 | 220 | 780 | 0 | 780 | −408 | 4.0 | 4,480 | 2,293 | 2,187 | 3.494 | 1.706 | 2.59 | 1.06 | 0.13 |
| 14-Nov | B | 5 | 380 | 220 | 840 | 0 | 840 | −413 | 4.1 | 4,480 | 2,453 | 2,027 | 3.763 | 1.702 | 1.82 | 1.06 | 0.13 |
| 14-Nov | C | 5 | 350 | 170 | 870 | 0 | 870 | −318 | 4.1 | 4,480 | 2,293 | 2,187 | 3.898 | 1.902 | 1.68 | 0.82 | 0.09 |
| 14-Nov | D | 5 | 320 | 130 | 920 | 0 | 920 | −372 | 4.1 | 4,480 | 1,920 | 2,560 | 4.122 | 2.355 | 1.54 | 0.62 | 0.06 |
| 14-Nov | E | 5 | 240 | 100 | 920 | 0 | 920 | −324 | 4.3 | 4,480 | 2,773 | 1,707 | 4.122 | 1.570 | 1.15 | 0.48 | 0.06 |
| 14-Nov | F | 5 | 50 | 25 | 810 | 0 | 810 | −329 | 4.0 | 3,307 | 2,080 | 1,227 | 2.679 | 0.994 | 0.24 | 0.12 | 0.03 |
| 15-Nov | A | 5.5 | 450 | 230 | 1120 | 25 | 1095 | −400 | 4.0 | 3,307 | 3,787 | (480) | 3.621 | −0.525 | 1.96 | 1.00 | −0.44 |
| 15-Nov | B | 5.5 | 450 | 235 | 1180 | 35 | 1145 | −384 | 4.0 | 3,307 | 3,253 | 54 | 3.787 | 0.061 | 1.96 | 1.03 | 3.82 |
| 15-Nov | C | 5.5 | 250 | 130 | 640 | 0 | 640 | −278 | 4.0 | 3,307 | 3,520 | (213) | 2.116 | −0.136 | 1.09 | 0.57 | −0.95 |
| 15-Nov | E | 5.5 | 455 | 225 | 1160 | 0 | 1160 | −435 | 4.0 | 3,307 | 3,467 | (160) | 3.836 | −0.185 | 1.99 | 0.98 | −1.21 |
| 15-Nov | F | 5.5 | 430 | 235 | 1160 | 0 | 1160 | −312 | 4.0 | 3,307 | 3,413 | (106) | 3.836 | −0.123 | 1.88 | 1.03 | −1.91 |
| 16-Nov | A | 5 | 380 | 190 | 1020 | 27 | 993 | −414 | 4.0 | 4,693 | 3,627 | 1,066 | 4.660 | 1.059 | 1.82 | 0.91 | 0.18 |
| 5-Dec | A | 4.5 | 200 | 110 | 500 | 35 | 465 | −439 | 4.0 | 4,267 | 4,160 | 107 | 1.984 | 0.050 | 1.07 | 0.59 | 2.21 |
| 18-Nov | A | 5 | 360 | 190 | 200 | 0 | 200 | −423 | 4.0 | 3,680 | 5,227 | (1,547) | 0.736 | −0.309 | 1.73 | 0.91 | −0.61 |
| 21-Nov | A | 4 | 320 | 170 | 800 | 40 | 760 | −429 | 4.0 | 3,493 | 3,680 | (187) | 2.656 | −0.142 | 1.92 | 1.02 | −1.20 |
| 22-Nov | A | 3.75 | 285 | 190 | 725 | 21 | 704 | −432 | 4.0 | 4,107 | 2,293 | 1,813 | 2.891 | 1.277 | 1.82 | 1.22 | 0.15 |
| 29-Nov | A | 4.25 | 310 | 155 | 750 | 24 | 726 | −439 | 4.0 | 5,013 | 3,520 | 1,493 | 3.640 | 1.084 | 1.75 | 0.88 | 0.14 |
| 2-Dec | A | 3.75 | 250 | 120 | 660 | 26 | 634 | −438 | 4.0 | 4,587 | 3,893 | 694 | 2.908 | 0.440 | 1.60 | 0.77 | 0.27 |
| 6-Dec | A | 3 | 150 | 75 | 540 | 0 | 540 | −441 | 4.0 | 4,853 | 3,093 | 1,760 | 2.621 | 0.950 | 1.20 | 0.60 | 0.08 |
| 17-Nov | A | 5.5 | 300 | 160 | 1010 | 30 | 980 | −414 | 4.0 | 4,907 | 3,520 | 1,387 | 4.809 | 1.359 | 1.31 | 0.70 | 0.12 |
| averages | | 4.81 | 324 | 164 | 830 | 13 | 817 | −392 | 4.0 | 4,092 | 3,213 | 879 | 3.344 | 0.718 | 1.61 | 0.82 | 0.23 |
| 16-Nov | B | 5 | 400 | 200 | 1125 | 45 | 1080 | −397 | 4.5 | 4,693 | 3,520 | 1,173 | 5.068 | 1.267 | 1.92 | 0.96 | 0.16 |
| 16-Nov | D | 5 | 400 | 165 | 960 | 60 | 900 | −360 | 4.5 | 4,693 | 3,573 | 1,120 | 4.224 | 1.008 | 1.92 | 0.79 | 0.16 |
| 16-Nov | E | 5 | 490 | 240 | 1100 | 72 | 1028 | −324 | 4.5 | 4,693 | 3,413 | 1,280 | 4.824 | 1.315 | 2.35 | 1.15 | 0.18 |
| 1-Dec | B | 3.5 | 500 | 260 | 570 | 45 | 525 | −415 | 4.5 | 5,173 | 3,680 | 1,493 | 2.716 | 0.784 | 3.43 | 1.78 | 0.33 |
| 6-Dec | B | 3 | 470 | 240 | 650 | 40 | 610 | −411 | 4.5 | 4,853 | 3,360 | 1,493 | 2.960 | 0.911 | 3.76 | 1.92 | 0.26 |
| 21-Nov | B | 4 | 560 | 300 | 930 | 50 | 880 | −397 | 4.5 | 3,493 | 3,147 | 346 | 3.074 | 0.305 | 3.36 | 1.80 | 0.98 |
| 2-Dec | B | 3.75 | 640 | 320 | 830 | 50 | 780 | −407 | 4.5 | 4,587 | 3,413 | 1,174 | 3.578 | 0.915 | 4.10 | 2.05 | 0.35 |
| 17-Nov | B | 5.5 | 450 | 220 | 1165 | 50 | 1115 | −406 | 4.5 | 4,907 | 2,933 | 1,974 | 5.471 | 2.201 | 1.96 | 0.96 | 0.10 |
| 18-Nov | B | 5 | 390 | 220 | 860 | 42 | 818 | −406 | 4.5 | 3,680 | 2,960 | 720 | 3.010 | 0.589 | 1.87 | 1.06 | 0.37 |
| 22-Nov | B | 3.75 | 585 | 395 | 835 | 50 | 785 | −397 | 4.5 | 4,107 | 2,720 | 1,387 | 3.224 | 1.089 | 3.74 | 2.53 | 0.36 |
| 29-Nov | B | 4.25 | 620 | 320 | 920 | 42 | 878 | −410 | 4.5 | 5,013 | 3,307 | 1,707 | 4.402 | 1.498 | 3.50 | 1.81 | 0.21 |
| 5-Dec | B | 4.5 | 390 | 190 | 750 | 37 | 713 | −417 | 4.5 | 4,267 | 3,840 | 427 | 3.042 | 0.304 | 2.08 | 1.01 | 0.62 |
| 16-Nov | F | 5 | 400 | 200 | 1082 | 93 | 989 | −324 | 4.5 | 4,693 | 3,093 | 1,600 | 4.641 | 1.582 | 1.92 | 0.96 | 0.13 |
| 16-Nov | C | 5 | 400 | 200 | 950 | 74 | 876 | −325 | 4.6 | 4,693 | 2,933 | 1,760 | 4.111 | 1.541 | 1.92 | 0.96 | 0.13 |
| averages | | 4.45 | 478 | 248 | 909 | 54 | 856 | −385 | 4.5 | 4,539 | 3,278 | 1,261 | 3.883 | 1.079 | 2.58 | 1.34 | 0.23 |

### SELECTED CITATIONS AND BIBLIOGRAPHY

Brosseau, J. D. and J. E. Zajic. 1982a. Continuous Microbial Production of Hydrogen Gas. Int. J. Hydrogen Energy 7(8): 623-628.
Brosseau, J. D. and J. E. Zajic. 1982ba. Hydrogen-gas Production with Citrobacter intermedius and Clostridium pasteurianum. J. Chem. Tech. Biotechnol. 32:496-502.
Cheresources Online Chemical Engineering Information, http://www.cheresources.com/heat_transfer_basics.shtml.
Iyer, P., M. A. Bruns. H. Zhang. S. Van Ginkel, and B. E. Logan. 2004. Hydrogen gas production in a continuous flow bioreactor using heat-treated soil inocula. Appl. Microbiol. Biotechnol. 89(1):119-127.
Kalia, V. C., et al. 1994. Fermentation of biowaste to H2 by Bacillus licheniformis. World Journal of Microbiol & Biotechnol. 10:224-227.
Kosaric, N. and R. P. Lyng. 1988. Chapter 5: Microbial Production of Hydrogen. In Biotechnology, Vol. 6B. editors Rehm & Reed. pp 101-137. Weinheim: Vett.
Logan, B. E., S.-E. Oh, I. S. Kim, and S. Van Ginkel. 2002. Biological hydrogen production measured in batch anaerobic respirometers. Environ. Sci. Technol. 36(11):2530-2535.
Logan, B. E. 2004. Biologically extracting energy from wastewater: biohydrogen production and microbial fuel cells. Environ. Sci. Technol., 38(9):160A-167A
Madigan, M. T., J. M. Martinko, and J. Parker. 1997. Brock Biology of Microorganisms, Eighth Edition, Prentice Hall, New Jersey. Nandi, R. and S. Sengupta. 1998. Microbial Production of Hydrogen: An Overview. Critical Reviews in Microbiology, 24(1):61-84.
Noike et al. 2002. Inhibition of hydrogen fermentation of organic wastes by lactic acid bacteria. International Journal of Hydrogen Energy. 27:1367-1372
Oh, S.-E., S. Van Ginkel, and B. E. Logan. 2003. The relative effectiveness of pH control and heat treatment for enhancing biohydrogen gas production. Environ. Sci. Technol., 37(22):5186-5190.
Prabha et al. 2003. H₂-Producing bacterial communities from a heat-treated soil Inoculum. Appl. Microbiol. Biotechnol. 66:166-173
Wang et al. 2003. Hydrogen Production from Wastewater Sludge Using a Clostridium Strain. J. Env. Sci. Health. Vol. A38(9):1867-1875
Yokoi et al. 2002. Microbial production of hydrogen from starch-manufacturing wastes. Biomass & Bioenergy; Vol. 22 (5):389-396.

We claim:

1. An apparatus for producing hydrogen from an organic feed material comprising:
a bioreactor adapted to receive therein the organic feed material to produce the hydrogen from microorganisms metabolizing the organic feed material,
means for heating the organic feed material before it is introduced into the bioreactor, wherein methanogens in the organic feed material are substantially killed or deactivated, and
means for removing the hydrogen from the bioreactor.

2. The apparatus of claim 1, wherein the heating means is a heat exchanger associated with a heat waste source.

3. The apparatus of claim 2, wherein the heat exchanger is selected from the group consisting of a gas/liquid heat exchanger and a liquid/liquid heat exchanger.

4. The apparatus of claim 1, including a container for holding the organic feed material.

5. The apparatus of claim 4, wherein the container is selected from the group consisting of a reservoir or an equalization tank.

6. The apparatus of claim 3, including treatment means for treating the organic feed material.

7. The apparatus of claim 7, including an electronic controller having at least one microprocessor adapted to process signals from a one or a plurality of devices providing water parameter information, wherein the electronic controller is connected to the at least one actuatable terminal and is arranged to control the operation of the heat exchanger and the temperature of any contents therein.

8. The apparatus of claim 4, including a pump in combination with a passage to provide a controlled flow of the organic feed material.

9. An industrial facility that produces byproducts of an organic feed material and heat, the industrial facility being adapted to convert the organic feed material into hydrogen, the industrial facility comprising a bioreactor adapted to receive the organic feed material and to produce the hydrogen from microorganisms metabolizing the organic feed material, means for heating the organic feed material before it is introduced into the bioreactor, wherein methanogens in the organic feed material are substantially killed or deactivated, and means for removing the hydrogen from the bioreactor.

10. The industrial facility of claim 9, wherein the industrial facility includes a juice industrial facility.

11. The industrial facility of claim 9, wherein the industrial facility includes a sewage treatment plant.

12. The industrial facility of claim 9, wherein the heat is conveyed to a heat exchanger and thereafter as the means for heating the organic feed material.
